(19) United States
(12) Patent Application Publication
(10) Pub. No.: US 2005/0273117 A1
(43) Pub. Date: Dec. 8, 2005

(54) Ring with blood sampling for dna analysis

(76) Inventor: Bruno Teychene, Mouzieys-Teulet (FR)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/532,569

(22) PCT Filed: Oct. 21, 2003
(86) PCT No.: PCT/EP03/50740

§ 371 Date: Apr. 21, 2005

(30) Foreign Application Priority Data
Oct. 22, 2002 (FR) 02/13134

(51) Int. Cl. A61B 17/00

(52) U.S. Cl. 606/116

(57) ABSTRACT

The invention concerns a ring for marking and identifying animals comprising a male tag with a punch passing through the animal's ear and penetrating into a hollow head (2) of a female tag (1) under the action of a clamp. The invention is characterised in that one of the male or female tags is provided with a means (7), lateral to label and detachable therefrom, for sampling biological material extracted from the animal's ear wall when it is pieced by the punch, said sampling means including an absorbent material (9/13), and in that said means (7) is linked to one of the jaws (3) of the clamp and the opening of the clamp jaws and/or its removal from the animal's ear separates the sampling means (7) from the tag concerned (1).

The present invention relates to the field of marking and identifying productive livestock.

Since the marking of animals by means of tags attached to their ears is mandatory, a means of taking a sample of organic material that can be retrieved and stored to identify the animal subsequently, in particular by its DNA, is known.

Known ear tags comprise a tag panel with a hollow head, the so-called female tag panel, and a tag panel with a punch, the so-called male tag panel, the punch penetrating into the hollow head after having passed through the animal's ear wall under the action of a clamp, wherein one jaw receives the female tag panel and the other the male tag panel.

Previous patents, notably WO 99/61882, DE19740429 and FR 0104570, are based on the sampling of biological material by a cutting die means borne by one of the jaws of the clamp, said means being separated from the clamp for storage.

This known technique has the advantage of taking the sample at the same time as the tag is fitted and of allowing the biological material sampled to be removed after the fitting of the tag and the withdrawal of the clamp.

Patent WO 01/87054 describes a device for taking a specimen for cattle identification that is no longer attached to the clamp but is fixed to one of the ear tag panels.

More precisely, this sampling device is a strip of absorbent material glued both to the tag panel bearing the punch, and around said punch.

This patent uses the absorbent capacity of the material to take a blood and/or cell sample or a sample of the mucous membranes of the animal.

According to this patent, the sampling operation is carried out at the same time as the tag is fitted, but the removal of the specimen carrier consisting of the strip of absorbent material must be carried out after the clamp has been removed by unsticking the strip while the tag is fastened to the animal, which is not easy.

Moreover, as it is glued to the base of the male punch, the absorbent strip is at a distance from the point at which the ear is pierced, and the sample taken may be quantitatively insufficient or even non-existent.

It became apparent, nevertheless, that taking a drop of blood using an absorbent material, as was performed in neonatal tests before the invention described in patent WO 01/87054, could have application in the field concerned, provided that the conditions of use corresponded to those suitable for the particular case of applying tags to animals'ears, which optimally require that the operation should be carried out instantaneously and not in stages.

This is one of the goals of the present invention, which the invention achieves by use of an ear tag that facilitates the sampling operation, together with a special clamp.

To this end, the ear tag for marking and identifying animals according to the invention, comprising a male tag panel with a punch that perforates the animal's ear and, after having passed through the ear, penetrates into the hollow head of a female tag panel under the action of a clamp, is characterised in that one of the male or female tag panels is provided with a means, lateral to the tag panel and separable therefrom, for sampling biological material extracted from the animal's ear when it is pierced by the punch, said sampling means comprising an absorbent material, and in that said means is linked to one of the jaws of the clamp and the opening of the clamp jaws and/or its removal from the animal's ear separates the sampling means from the tag concerned.

According to another embodiment, the ear tag is characterised in that the sampling means is attached to the female tag panel.

According to another embodiment, the tag is characterised in that the sampling means comprises an attachment to be fixed to one of the jaws of the clamp and an absorbent material.

The ear tag is also characterised in that the absorbent material is disposed transversely to the direction of penetration of the male punch through the ear and to the axis of the hollow head of the female tag panel.

The ear tag is also characterised in that the absorbent material is a sampling strip, wherein one end is fixed to the attachment and the other free end is transversal to the direction of penetration of the punch through the animal's ear.

The ear tag is also characterised in that the absorbent material is connected by one of its ends transversely to the interior of the hollow head of the female tag panel and by its other end to an attachment to be fixed to the jaw of the clamp.

The clamp enabling the fitting of the ear tag according the invention, of which it forms an integral part, is characterised in that one of the jaws of the clamp is provided with a fixing means for the attachment to which the absorbent material is linked.

Further advantages and characteristics of the invention will become apparent on reading the following description of forms of embodiment of the invention given as non-limiting examples and illustrated by the attached drawings, wherein:

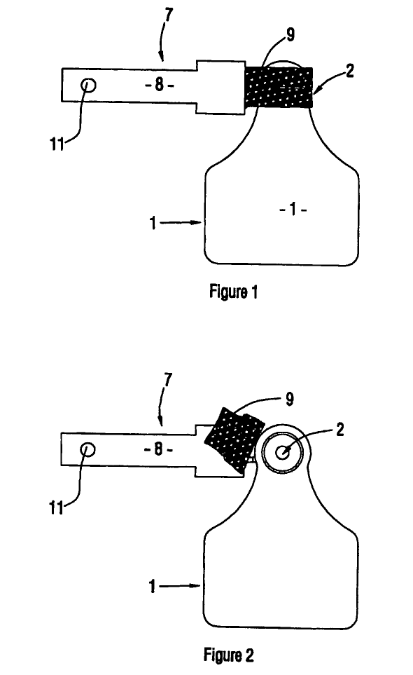

FIGS. 1 to 3 show a form of embodiment of an ear tag panel according to the invention;

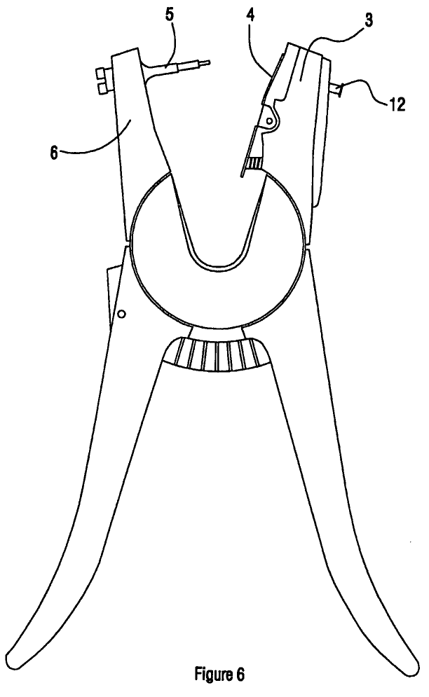

FIGS. 4-5 show a further form of embodiment of the ear tag panel according to the invention;

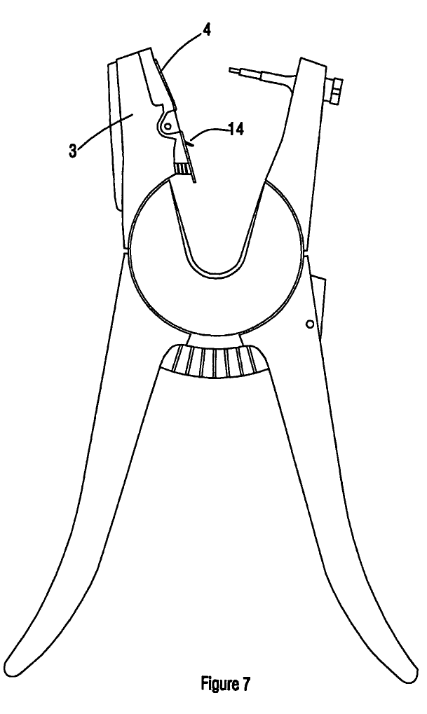

FIGS. 6 and 7 show two forms of embodiment of the clamp according to the invention for fitting ear tags according to the invention;

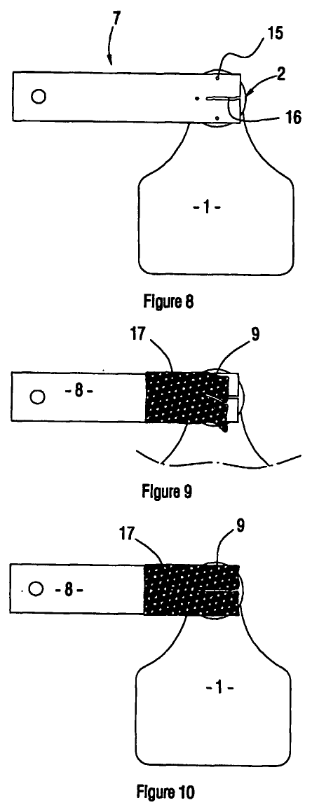

FIGS. 8, 9 and 10 show a further form of embodiment of the ear tag panel according to the invention;

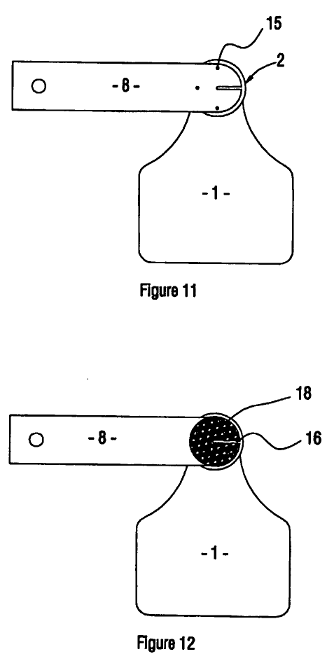

FIGS. 11 and 12 show a further form of embodiment of the tag panel according to the invention.

In the attached diagrams, FIGS. 1 to 5 show a so-called female tag panel 1, from an ear tag of known type comprising a male tag panel, not shown, with a pin in the form of a punch that pierces the animal's ear wall and is housed in the hollow head of the female tag panel.

Reference 1 designates the tag panel that carries conventional markings.

Reference 2 designates the hollow head into which the punch of the male tag panel (not shown) is housed in permanent manner.

The hollow head of the female tag panel is mounted on the jaw 3 of the clamp (FIGS. 6-7) and is held there by a lever 4.

The punch of the female ¹[sic] tag panel is mounted on the prick punch 5 on the jaw 6 acting in opposition to the jaw 3.
¹TRANSLATOR'S NOTE: the French original uses the word female. This is probably meant to be male.

Bringing the jaws together with the ear wall between them causes the perforation of the latter and the permanent penetration of the male punch into the female head, the tag being fixed to the ear in a single operation.

Opening the clamp frees the animal's ear, now fitted with the tag.

According to the invention, either of the male or female tag panels is provided with a sampling means 7 that is attached to the panel in such a way that it can be separated from it when the tag is fixed to the animal's ear and the clamp is removed from the ear.

The sampling means lateral to the tag panel and separable from it comprises an attachment 8 to which is fixed, or which bears upon it, an absorbent material 9.

The attachment 8 is linked to one of the jaws 3/6 of the clamp for fitting the tag.

The absorbent material is disposed transversely to the direction of penetration of the punch of the male tag panel towards and into the hollow head of the female tag panel so that, when the ear wall is pierced, it collects the blood or the biological material removed from the ear.

The sampling means is disposed partly on the tag panel and partly outside the tag panel. The fixing onto the tag panel of the sampling means 7, consisting of the attachment 8 and the absorbent material, is carried out in a non-permanent way so that when the tag is fitted by opening the clamp to which the attachment 8 is fixed, the sampling means 7 is separated from the tag panel.

This separation is facilitated by the withdrawal and removal of the clamp from the animal's ear.

Preferably the sampling means is borne by the tag panel having the female head.

The absorbent material is disposed transversely to the axis of the female head, either at the mouth of its opening or in its blind channel.

It is particularly advantageous that the absorbent material should be posterior to the ear wall, thus ensuring that it will be in contact with the biological material borne by the punch.

FIGS. 1 to 3 show a particular form of embodiment of the invention.

FIGS. 1/2 show the female tag panel seen from the side of the mouth of the head 2.

The attachment 8 consists of a strip of synthetic or other material fixed to the edge of the tag at the level of the female head and radial to said head.

The attachment is fixed in such a way as to allow the connection with the tag to be broken by the pulling action of the user who, after opening the clamp, removes it from the animal's ear.

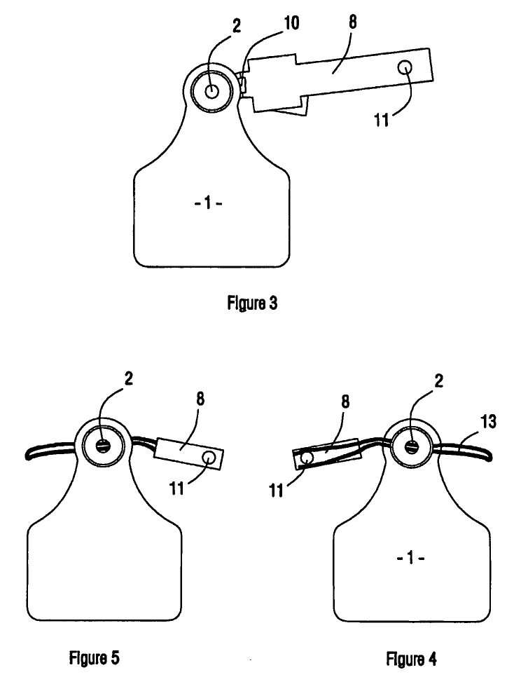

To this, end a break-line 10, for example a line of reduced thickness, is disposed on the attachment 8, level with where it joins to the known tag shown in FIG. 3.

In this form of embodiment, the absorbent material consists of a flexible strip of any known type that is fixed to the attachment strip by a part behind the break-line or area 10 and which is free forward of that area 10 above the opening of the female head.

The attachment 8 is provided with a means for fixing it to the jaw 3 of the clamp.

This means is a perforation 11 through which the folded attachment strip is locked onto a dog point 12 on the clamp jaw as shown in FIG. 6. In the example of embodiment of the invention shown in FIGS. 4 and 5, the hollow head of the female tag panel is provided with a transverse perforation across its diameter into which the absorbent material in the form of a wick 12 of appropriate cross-section is introduced, passing through the so-called head 2 and exiting on both sides.

On one side of the head, the wick is preferably fixed to the tag panel 1, for example by a glue spot 13.

On the other side of the head, the wick is fixed to an attachment 8 consisting of a strip of synthetic material or other provided with a perforation 11 that allows the attachment to be fixed to a hook 14 on the lever 4.

In FIGS. 8, 9 and 10 another form of embodiment of the tag panel according to the invention is shown.

In this form of embodiment, the strip of material making up the attachment is fixed on the opening 2 of the female head so that the punch of the male tag panel passes through it.

Fixing the attachment to the periphery of the opening of the female head is accomplished by weld points 15.

Advantageously, the part of the attachment that is over the opening of the female head is provided with a slit 16 allowing the punch to pass through. According to a further embodiment of the invention, the attachment is provided with an area 17 coated with adhesive to allow the fixing of the strip 9 of absorbent material, said area being behind the part that covers the opening of the female head, and said part being slit at 16.

This strip 9 of absorbent material is glued on the area 17 of the attachment and extends over the opening of the hollow head that it covers.

It can be provided with a slit corresponding to the slit 16 to allow the punch to pass through.

FIG. 10 shows the position of the strip 9 glued to one end of the attachment strip 8 and FIG. 9 shows that the other part of the sampling strip 9 is free.

FIGS. 11 and 12 show a further of embodiment of the invention in which the attachment strip 8 is fixed by weld points 15 onto the periphery of the opening of the hollow female head.

The area of the attachment strip 8 that covers the opening of the female head is preferably slit at 16 to facilitate the punch passing through.

The area of the strip covering the female head is provided with an adhesive and the absorbent material 9 consists of a preferably circular pad 18 glued over that area.

The absorbent material 9 is preferably also slit at 16.

In the forms of embodiment of the invention shown in FIGS. 8 to 12, the fixing of the strip making up the attachment by weld points 15 is designed to break, thus enabling the separation of the tag panel from the sampling means after it has been attached to the ear.

Advantageously, the male tag panel, the female tag panel and the attachment 8 are provided with identical numerical, alphabetical or bar-coded markings, for example, for identification.

1. Ear tag for marking and identifying animals comprising a male tag panel with a punch that, in use, perforates the animal's ear and penetrates into a hollow head of a female tag panel under the action of an applicator tool, one of the male or female tag panels is provided with a sampling device, lateral to the tag panel and separable therefrom, for sampling biological material extracted from the ear of the animal when it is pierced by the punch, said sampling device comprising an absorbent material, and said sampling device is linked to one of the jaws of the applicator tool and the opening of the clamp jaws and/or its removal from the animal's ear separates the sampling device from the tag panel concerned.

2. Ear tag according to claim 1, wherein the sampling device is attached to the female tag panel.

3. Ear tag according to claim 1 wherein the sampling device comprises an attachment to be fixed on one of the jaws of the applicator tool.

4. Ear tag according to claim 1, wherein absorbent material is disposed transversely to the direction in which, in use the male punch penetrates through the animal's ear and to the axis of the hollow head of the female tag panel, the sampling material being posterior to the ear wall.

5. Ear tag according to claim 1 wherein the absorbent material is a sampling strip one end of which is fixed to the attachment linked to the applicator tool and the other is transversal to the direction of penetration of the punch through the animal's ear.

6. Ear tag according to claim 1 wherein the absorbent material is connected by one of its ends transversely to the interior of the hollow head of the female tag panel and by its other end to an attachment to be fixed to the jaw of the applicator tool.

7. Applicator tool enabling the fitting of the ear tag of claims 3 wherein one of the jaws of the applicator tool is provided with a fixing arrangement for the attachment to which the absorbent material is linked.

8. Ear tag according to claim 1 wherein the absorbent material is disposed transversely to the axis of the hollow head of the female tag panel.

9. Ear tag according to claim 3 wherein the attachment is fixed onto the periphery of an opening of the hollow head of the female tag panel.

10. Ear tag according to claim 9, wherein the attachment and the sampling device are slit over the opening of the hollow head of the female tag panel.

11. Ear tag according to claim 3 wherein the male tag panel, the female tag panel and the attachment are provided with identical symbols for marking and identification.

12. Ear tag for marking and identifying animals the ear tag including a male tag component with a punch that, in use, perforates the animal's ear and penetrates into a hollow head of a female tag component under the action of an applicator tool, the female tag component is provided with a sampling device, lateral to the female component and separable therefrom, for sampling biological material extracted from the ear of the animal when it is pierced by the punch. said sampling device having an absorbent material, and an attachment to fix the sampling device to one of the jaws of the applicator tool and upon opening of the jaws and/or its removal from the animal's ear separates the sampling device from the female tag component.

13. Ear tag according to claim 12, wherein the absorbent material is disposed transversely to the direction in which, in use, the male punch penetrates through the animal's ear and to the axis of the hollow head of the female tag component, the sampling material being posterior to the ear wall.

14. Ear tag according to claim 12 or 13 wherein the absorbent material is a sampling strip, one end of which is fixed to the attachment and the other is located transverse to the direction of penetration of the punch through the animal's ear.

15. Ear tag according to claim 14 wherein the absorbent material is connected by one of its ends transversely to the interior of the hollow head of the female tag panel and by its other end to said attachment.

16. Ear tag according to claim 14 wherein the attachment is fixed onto the periphery of the hollow head of the female tag component.

17. Ear tag according to claim 16 wherein the attachment and sampling device are slit over an opening of the hollow head of the female tag component.

18. Ear tag according to claim 15 wherein the attachment is fixed onto the periphery of the hollow head of the female tag component.

19. Ear tag according to claim 18 wherein the attachment and the sampling device are slit over an opening of the hollow head of the female tag component.

20. Ear tag according to claim 12 or 13 wherein the male tag component, the female tag component each include a panel, the panels and the attachment being each provided with identical symbols for marking and identification.
